(19) United States
(12) Patent Application Publication
(10) Pub. No.: US 2008/0063730 A9
(43) Pub. Date: Mar. 13, 2008

(54) Methods for prophylactic and therapeutic nutritional supplementation

(76) Inventor: John A. Giordano, West Orange, NJ (US)

Correspondence Address: DON J. PELTO;Sheppard, Mullin, Richter & Hampton LLP, 1300 I STREET, NW, 11TH FLOOR EAST, WASHINGTON, DC, 20006, US

(21) Appl. No.: 11/201,285

(22) Filed: Aug. 11, 2005

Related U.S. Application Data
(63) Continuation-in-part of application No. 10/916,534, filed on Aug. 12, 2004.
(63) Continuation-in-part of application No. 10/964,754, filed on Oct. 15, 2004.
(63) Continuation-in-part of application No. 10/901,054, filed on Jul. 29, 2004.
(63) Continuation-in-part of application No. 11/049,643, filed on Feb. 4, 2005.

(51) Int. Cl. A61K 31/714; A61K 31/525; A61K 31/51; A61K 31/455; A61K 31/4415; A61K 31/375; A61K 31/4188; A61K 31/385; A61K 31/355; A61K 33/34; A61K 33/32; A61K 33/26; A61K 33/24

(52) U.S. Cl. 424/638; 424/643; 424/646; 424/639; 424/702; 424/655; 514/52; 514/250; 514/251; 514/168; 514/276; 514/350; 514/355; 514/393; 514/725; 514/763; 514/440; 514/458; 514/356

(57) ABSTRACT

The present invention relates to methods for prophylactic nutritional supplementation and therapeutic nutritional supplementation. Specifically, the method involves administering to an individual a composition comprising vitamin A, vitamin E, vitamin D, vitamin C, thiamine, riboflavin, niacin, folic acid, pyridoxine, biotin, pantothenic acid, cobalamin, magnesium, manganese, zinc, selenium, chromium, copper, iron, alpha lipoic acid, lutein, and lycopene to prevent, treat and/or alleviate the occurrence or negative effects of vitamin and mineral deficiencies and to prevent, treat and/or alleviate the occurrence or negative effects of various disease states including cancer, cardiovascular disease and anemia.

### FIELD OF THE INVENTION

The present invention relates to methods for using compositions for prophylactic nutritional supplementation and therapeutic nutritional supplementation to prevent, treat and/or alleviate the occurrence or negative effects of vitamin and mineral deficiencies in premenopausal women, dieters, high-impact endurance athletes, infants, children, adolescents, and pregnant women and to prevent, treat and/or alleviate the occurrence or negative effects of various disease states including cancer, cardiovascular disease and anemia.

### BACKGROUND OF THE INVENTION

Nutrition plays a critical role in maintaining good health. Proper nutrition prevents dietary deficiencies, and also protects against the development of disease. Proper nutrition plays an increasingly important role as the body faces physiological stress including dieting, high-impact exercise, adolescent growth, menstruation and pregnancy.

Thus, nutritional supplementation serves a vital role in protecting against poor nutrition and disease. More specifically, nutritional supplementation may provide the necessary vitamins, minerals, and other nutrients that might otherwise be lacking in the diet, and provide the nutritional defense against disease development including cancer, cardiovascular disease, and anemia. The invention herein provides for methods designed to optimize health and wellness through supplementation to prevent, treat and/or alleviate the occurrence or negative effects of vitamin and mineral deficiencies including iron and folic acid deficiency.

### SUMMARY OF THE INVENTION

The present invention provides methods of utilizing compositions for both prophylactic and therapeutic nutritional supplementation, for use in physiologically stressful conditions and for the treatment and prevention of various diseases including cancer, cardiovascular disease, and anemia. Specifically, the present invention relates to novel compositions of vitamins and minerals that can be used to supplement the nutritional deficiencies observed in patients with physiological stress including dieting, high-impact exercise, adolescent growth, menstruation and pregnancy and/or dietary restrictions. In addition, the compositions may be used to treat the nutritional deficiencies of patients suffering from anemia.

The methods of the present invention comprise compositions that include various vitamins and minerals that improve the nutritional state of a patient; these compositions preferably may be used therapeutically or prophylacticly. The vitamins of the present invention may preferably comprise one or more of vitamin A, vitamin E, vitamin D3, vitamin C, thiamine, riboflavin, niacin, folic acid, pyridoxine, biotin, pantothenic acid, and cyanocobalamin. The minerals of the present invention may preferably include one or more of magnesium, zinc, selenium, chromium, copper and iron. In addition, the present invention may preferably comprise other nutritional elements, such as alpha lipoic acid, lutein, and/or lycopene. In addition, the present invention may be substantially free of other added vitamins and added minerals.

In a preferred embodiment, the methods of the present invention may include folic acid in the form of vitamin B9, folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

In a preferred embodiment, the methods of the present invention may comprise vitamin E in the form of d-alpha tocopheryl succinate, vitamin D in the form of vitamin D3, thiamine in the form of thiamine mononitrate, niacin in the form of niacinamide, chromium in the form of chromium chloride, selenium in the form of sodium selenate or selenomethionine, zinc in the form of zinc oxide, vitamin A in the form of alpha-carotene or beta-carotene, magnesium in the form of magnesium oxide, copper in the form of cupric sulfate or gluconate, chromium in the form of picolinate, pantothenic acid in the form of calcium pantothenate, cobalamin in the form of cyanocobalamin, iron in the form of iron ferronyl, and pyridoxine in the form of pyridoxine HCL.

In one embodiment of the present invention, the methods may utilize compositions in a swallowable form. In another embodiment, the methods may utilize compositions substantially free of other added vitamins, added minerals, and added coenzymes. In another embodiment, the methods may utilize compositions comprising pharmaceutically acceptable carriers, such as one or more of binders, diluents, lubricants, glidants, colorants, emulsifiers, disintegrants, starches, water, oils, alcohols, preservatives and sugars.

In a preferred embodiment, the methods of the present invention may utilize compositions comprising about 375 IU to about 1125 IU vitamin A, about 62.5 IU to about 187.5 IU vitamin E, about 157.5 IU to about 472.5 IU vitamin D, about 187.5 mg to about 562.5 mg vitamin C, about 12.5 mg to about 37.5 mg thiamine, about 2.9 mg to about 6.3 mg riboflavin, about 17.5 mg to about 52.5 mg niacin, about 0.625 mg to about 1.875 mg folic acid, about 17.5 mg to about 52.5 mg pyridoxine, about 37.5 μg to about 112.5 μg biotin, about 2.5 mg to about 7.5 mg pantothenic acid, about 35 μg to about 105 μg cobalamin, about 17.5 mg to about 52.5 mg magnesium, about 17.5 mg to about 52.5 mg zinc, about 62.5 μg to about 187.5 μg selenium, about 75 μg to about 225 μg chromium, about 0.5 mg to about 1.5 mg copper, about 6.5 mg to about 19.5 mg iron, about 5 mg to about 15 mg alpha lipoic acid, about 3.5 mg to about 10.5 mg lutein, and about 1.25 mg to about 3.75 mg lycopene.

In another preferred embodiment, the methods of the present invention may utilize compositions comprising about 600 IU to about 900 IU vitamin A, about 100 IU to about 150 IU vitamin E, about 252 IU to about 378 IU vitamin D, about 300 mg to about 450 mg vitamin C, about 20 mg to about 30 mg thiamine, about 2.72 mg to about 4.08 mg riboflavin, about 28 mg to about 42 mg niacin, about 1.0 mg to about 1.5 mg folic acid, about 28 mg to about 42 mg pyridoxine, about 50 μg to about 100 μg biotin, about 4 mg to about 6 mg pantothenic acid, about 56 μg to about 84 μg cobalamin, about 28 mg to about 42 mg magnesium, about 28 mg to about 42 mg zinc, about 100 μg to about 150 μg selenium, about 120 μg to about 170 μg chromium, about 0.8 mg to about 1.2 mg copper, about 10.4 to about 15.6 mg iron, about 8 mg to about 12 mg alpha lipoic acid, about 5.6 mg to about 8.4 mg lutein and about 2 mg to about 3 mg lycopene.

In yet another preferred embodiment, the methods of the present invention may utilize compositions comprising about 675 IU to about 825 IU vitamin A, about 112.5 IU to about 137.5 IU vitamin E, about 283.5 IU to about 346.5 IU vitamin D, about 337.5 mg to about 412.5 mg vitamin C, about 22.5 mg to about 27.5 mg thiamine, about 3.06 mg to about 3.74 mg riboflavin, about 31.5 mg to about 37.5 mg niacin, about 1.125 mg to about 1.375 mg folic acid, about 31.5 mg to about 37.5 mg pyridoxine, about 67.5 μg to about 82.5 μg biotin, about 4.5 mg to about 5.5 mg pantothenic acid, about 63 μg to about 77 μg cobalamin, about 31.5 mg to about 37.5 mg magnesium, about 32.5 mg to about 37.5 mg zinc, about 112.5 μg to about 137.5 μg selenium, about 135 μg to about 165 μg chromium, about 0.9 mg to about 1.1 mg copper, about 11.7 to about 14.3 mg iron, about 9 mg to about 11 mg alpha lipoic acid, about 6.3 mg to about 7.7 mg lutein, and about 2.25 mg to about 2.75 mg lycopene.

In another preferred embodiment, the methods of the present invention may utilize compositions comprising about 750 IU vitamin A, about 125 IU vitamin E, about 315 IU vitamin D, about 375 mg vitamin C, about 22 mg thiamine, about 3.4 mg riboflavin, about 35 mg niacin, about 1.25 mg folic acid, about 35 mg pyridoxine, about 75 μg biotin, about 5 mg pantothenic acid, about 70 μg cobalamin, about 35 mg magnesium, about 35 mg zinc, about 125 μg selenium, about 150 μg chromium, about 1.0 mg copper, about 13 mg iron, about 10 mg alpha lipoic acid, about 7 mg lutein, and about 2.5 mg lycopene.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION

It is understood that the present invention is not limited to the particular methodologies, protocols, fillers, and excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “subject,” as used herein, comprises any and all organisms and includes the term “patient.” “Subject” may refer to a human or any other animal.

The phrase “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “swallowable form” refers to any compositions that do not readily dissolve when placed in the mouth and may be swallowed whole without any chewing or discomfort. In one embodiment, may have a shape containing no sharp edges and a smooth, uniform and substantially bubble free outer coating.

The phrase, “no added vitamins or minerals” does not include trace amounts of vitamins or minerals added during the manufacturing, tableting, or mixing process. These trace amounts of vitamins or minerals are not therapeutically significant.

Proper nutrition is essential for maintaining health and preventing diseases. The compositions and methods of the present invention provide the means to optimize good health by utilizing vitamin, mineral, and antioxidant nutritional supplementation. More specifically, proper nutrition plays an increasingly important role as the body faces physiological stress including dieting, high-impact exercise, adolescent growth, menstruation and pregnancy. Additionally, nutritional supplementation may provide the necessary vitamins, minerals, and other nutrients that might otherwise be lacking in the diet, and provide the nutritional defense against disease development including cancer, cardiovascular disease and anemia. The invention herein provides for methods designed to optimize health and wellness through supplementation to prevent, treat and/or alleviate the occurrence or negative effects of vitamin and mineral deficiencies including iron and folic acid deficiency and anemia.

The methods of the present invention provide means to optimize good health by utilizing vitamin and mineral combinations that specifically aim to prevent, treat and/or alleviate the occurrence or negative effects vitamin and mineral deficiencies including iron and folic acid deficiency. The compositions and methods of the present invention may be administered to or directed to a subject such as a human or any other organism. Each of the added vitamins and minerals that can be included in the present invention, including vitamin A, vitamin E, vitamin D, vitamin C, thiamine, riboflavin, niacin, folic acid, pyridoxine, biotin, pantothenic acid, cobalamin, magnesium, zinc, selenium, about chromium, copper, iron, alpha lipoic acid, lutein, and lycopene, plays a specific role in preventing, treating and/or alleviating the occurrence or negative effects of iron and folic acid deficiency including anemia and various diseases including cancer and cardiovascular disease.

In a specific embodiment, vitamins and minerals that inhibit these compounds' beneficial effects may be specifically excluded from the compositions utilized by the methods of the present invention. Further, in another specific embodiment, other added vitamins and/or minerals can be excluded.

The methods of the present invention also may preferably include B-complex vitamins, which are critical for health as each is part of one or more coenzymes in metabolizing food properly. This class of vitamins is water-soluble nutrients, not stored significantly in the body. Importantly, the B-complex vitamins may help normalize homocysteine levels and metabolism. High homocysteine levels have been correlated directly with increased risk of atherosclerosis and other heart disease. Although the exact mechanism by which homocysteine contributes to heart disease is not fully understood, it may act as an endothelial irritant that promotes atherosclerosis by inducing endothelial dysfunction. B-complex vitamins are required for the proper function of the homocysteine metabolic pathway, thus maintaining adequate levels of these vitamins may assist in normalizing homocysteine levels and maintaining good health. The B-complex vitamins of the present compositions utilized by the methods may preferably include one or more of thiamin (B1), riboflavin (B2), niacin (B3), pantothenic acid, biotin, folic acid, pyridoxine (B6) and cobalamin (B12).

Thiamine (vitamin B1) plays a role in carbohydrate metabolism and neural function. It is a coenzyme for the oxidative decarboxylation of alpha-ketoacids (e.g., alpha-ketoglutarate and pyruvate) and for transketolase which is a component of the pentose phosphate pathway. Folate deficiency and malnutrition inhibit the activity of thiamine. RDA, at 123. One embodiment of the compositions utilized by the methods of the present invention may comprise thiamine in an amount ranging from about 12.5 mg to about 37.5 mg. In another embodiment of the invention, thiamine may be present in a range of about 30 mg to about 20 mg. In a further embodiment of the invention, thiamine may be present in a range of about 22.5 mg to about 27.5 mg. In a preferred embodiment, thiamine may be present in the amount of about 25 mg. In a preferred embodiment of the present invention, the form of thiamine is thiamine HCl.

Riboflavin (vitamin B2) is a component of two flavin coenzymes, flavin mononucleotide (FMN) and flavin adenine dinucleotide (FAD). These flavoenzymes are involved in a number of oxidation-reduction reactions including the conversion of pyridoxine and niacin. RDA, at 132. Flavoenzymes also play a role in a number of metabolic pathways such as citric acid cycle, amino acid deamination, purine degradation, and fatty acid oxidation and thus help to maintain carbohydrate, amino acid, and lipid metabolism. In one embodiment, the compositions utilized by the methods of the present invention may comprise riboflavin present in a range of about 2.9 mg to about 6.3 mg. In another embodiment of the invention, riboflavin may be present in a range of about 2.72 mg to about 4.08 mg. In another embodiment, riboflavin may be present in a range of about 3.06 mg to about 3.74 mg. In a further embodiment, riboflavin may be present in the amount of about 3. 4 mg.

Niacin, also called vitamin B3, is the common name for two compounds: nicotinic acid (also called niacin) and niacinamide (also called nicotinamide). Niacin is particularly important for maintaining healthy levels and types of fatty acids. Niacin is also required for the synthesis of pyroxidine, riboflavin, and folic acid. RDA, at 137. Administration of niacin may also produce a reduction in total cholesterol, LDL, and very low density lipoprotein (VLDL) levels; and an increase in high density lipoprotein (HDL) cholesterol levels. Nicotinamide adenine dinucleotide (NAD) and NAD phosphate (ADP) are active coenzymes of niacin. These coenzymes are involved in numerous enzymatic reactions such as glycolysis, fatty acid metabolism, and steroid synthesis. Henkin et al., 91 Am. J. Med. 239-46 (1991). One embodiment of the compositions and utilized by the methods of the present invention may comprise niacin in the amount ranging from about 17.5 mg to about 52.5 mg. In another embodiment, niacin may be present in a range of about 28 mg to about 42 mg. In a further embodiment, niacin may be present in the range of about 31.5 mg to about 37.5 mg. In a preferred embodiment, niacin may be present in the amount of about 35 mg. In a preferred embodiment of the invention, niacin may be present in the form of niacinamide.

Folic acid (vitamin B8), also called folate or methylfolate, is essential for the formation of red and white blood cells within bone marrow and also plays a role in heme formation. RDA, at 150. Folic acid in its active form, tetrahydrofolate, is a coenzyme that is involved in the transfer of methyl groups and it plays a role in DNA synthesis, purine synthesis, and amino acid synthesis, such as the conversion of glycine to serine and the transformation of homocysteine to methionine. The activation of folic acid requires a vitamin B12-dependent transmethylation and vitamin B12 is also necessary for folic acid delivery to tissues. Id. Folic acid is essential for proper nutrition in pregnant women. Folic acid has been shown to help reduce the risk of birth defects, specifically neural tube defects, for example, spinal bifida if taken before conception and during the first three months of pregnancy. Available at

http://www.betterhealth.vic.gov.au/bhcv2/bhcarticles.nsf/pages/Pregnancy_and_diet?OpenDo cument (Last visited 10 Aug. 2005);

http://www.ifglobal.org/spina_bifida.asp?lang=1&main=6&sub=6 (Last visited 10 Aug. 2005).

One embodiment of the compositions utilized by the methods of the present invention may comprise folic acid in an amount ranging from about 0.625 to about 1.875 mg. In another embodiment, folic acid may be present in an amount ranging from about 1.00 mg to about 1.50 mg. In a further embodiment, folic acid may be present in the range of about 1.125 mg to about 1.375 mg. In a preferred embodiment, folic acid may be present in the amount of about 1.25 mg.

Pyridoxine (vitamin B6) is another B-complex vitamin included in the compositions utilized by the methods described herein. The administration of pyridoxine may reduce the levels of homocysteine. Bostom et al., 49 Kidney Int. 147-52 (1996). The active forms of pyridoxine, pyridoxal-5′-phosphate (PLP) and pyridoxamine-5′-phosphate, are coenzymes for numerous enzymes and as such, are essential for gluconeogenesis, niacin formation, and erythrocyte metabolism. RDA, at 142-143. Pyridoxine is a coenzyme for both cystathionine synthase and cystathionase, enzymes that catalyze the formation of cysteine from methionine. Homocysteine is an intermediate in this process and elevated levels of plasma homocysteine are recognized as a risk factor for vascular disease. Robinson et al., 94 Circulation 2743-48 (1996). Hence, one embodiment of the compositions utilized by the methods of the present invention may comprise pyridoxine in an amount ranging from about 17.5 mg to about 52.5 mg. In another embodiment, pyridoxine may be present in a range of about 28 mg to about 42 mg. In a further embodiment, pyridoxine may be present in a range of about 31.5 mg to about 37.5 mg. In a preferred embodiment, pyridoxine may be present in an amount of about 35 mg. In another preferred embodiment of the invention, pyridoxine is in the form of pyridoxine HCl.

Biotin, another water-soluble B-complex vitamin, acts a coenzyme for a number of carboxylases, and thus has an important role in gluconeogenesis, fatty acid metabolism, and amino acid metabolism. RDA, at 166. For example, biotin serves as a carboxyl carrier for pyruvate carboxylase, which is involved in gluconeogenesis; acetyl CoA carboxylase, which is involved in fatty acid synthesis; and propionyl-CoA carboxylase, which is involved in glucose production. Researchers believe that biotin inhibits the effects of uremic toxins on tubulin polymerizaton. Braguer et al., 57 Nephron 192-96 (1991). Hence, one embodiment of the compositions utilized by the methods of the present invention may comprise biotin in an amount ranging from about 37.5 μg to about 112.5 μg. In another embodiment, biotin may be present in a range of about 50 μg to about 100 μg. In a further embodiment, biotin may be present in a range of about 67.5 μg to about 82.5 μg. In a preferred embodiment, biotin may be present in an amount of about 75 μg.

Pantothenic acid (vitamin B5) is a component of both the coenzyme A macromolecule and the acyl-carrier protein. These coenzymes function as carriers for acyl groups and are required for the synthesis of fatty acids, cholesterol, steroid hormones, and neurotransmitters. The coenzyme A complex also has a major role in the acetylation and acylation of numerous proteins. RDA, at 169. One embodiment of the compositions utilized by the methods of the present invention may comprise pantothenic acid in an amount ranging from about 2.5 mg to about 7.5 mg. In another embodiment, pantothenic acid may be present in a range of about 4 mg to about 6 mg. In a further embodiment, pyridoxine may be present in a range of about 4.5 mg to about 5.5 mg. In a preferred embodiment, pantothenic acid may be present in an amount of about 5 mg. In another preferred embodiment of the invention, pantothenic acid may be in the form of calcium pantothenate.

Cobalamin (vitamin B12), another important vitamin included in the compositions utilized by the methods described herein, can be converted to the active coenzymes, methylcobalamin and 5′-deoxyadenosylcobalamin. These coenzymes are necessary for folic acid metabolism, conversion of coenzyme A, and myelin synthesis. For example, methylcobalamin catalyzes the demethylation of a folate cofactor, which is involved in DNA synthesis. A lack of demethylation may result in folic acid deficiency. RDA, at 159-160. Deoxyadenosylcobalamin is the coenzyme for the conversion of methylmalonyl-CoA to succinyl-CoA, which plays a role in the citric acid cycle. Importantly, cobalamin, along with pyridoxine and folic acid in implicated in the proper metabolism of homocysteine. Cobalamin is available as cyanocobalamin, methylcobalamin, hydroxocobalamin, adenosylcobalamin, and hydroxycyanocobalamin.

One embodiment of the compositions utilized by the methods of the present invention may comprise cobalamin in an amount ranging from about 35 μg to about 105 μg. In another embodiment, cobalamin may be present in an range of about 56 μg to about 84 μg. In a further embodiment, cobalamin may be present in a range of about 63 μg to about 77 μg. In a preferred embodiment, cobalamin may be present in an amount of about 70 μg. In another preferred embodiment of the invention, cobalamin may be in the form of cyanocobalamin.

Vitamin D may preferably be a component of the compositions utilized by the methods of the present invention. Vitamin D is a fat-soluble “hormone like” substance essential for healthy bones. This vitamin increases the absorption of calcium and phosphorous from the gastrointestinal tract, and improves essential mineral resorption into bone tissue. Vitamin D can be converted to its active form from exposure of the skin to sunlight. This fact is among the reasons why vitamin D deficiency is common in the elderly, notably the institutionalized, who spend little or no time out of doors. Deficiencies lead to increased bone turnover and loss, and when severe, osteomalacia or softening of the bones. Supplementation with vitamin D has been shown to moderately reduce bone loss, increase serum 25-hydroxyvitamin D, and decrease serum parathyroid hormone levels. Dawson-Hughes et al., 337 New Eng. J. Med. 670-76 (1997); Lips et al., 86 J. Clin. Endocrinol. Metab. 1212-21 (2001).

Preferably, the vitamin D of the compositions utilized by the methods of the present invention is vitamin D3. In the body, vitamin D3 is produced when its precursor is exposed to ultraviolet irradiation (e.g., sunlight) and then hydroxylated in the liver to form 25-hydroxyvitamin D3, the major form of vitamin D in the circulation. This form of the vitamin may be hydroxylated again in the kidney, yielding 1,25 hydroxyvitamin D3, the most potent form of vitamin D. As noted above, vitamin D3 plays a role in the maintenance of calcium and phosphorus homeostasis, but it is also active in cell differentiation and immune function.

One embodiment of the compositions utilized by the methods of the present invention may comprise vitamin D in an amount ranging from about 157.5 IU to about 462.5 IU. In Another embodiment of the invention comprises vitamin D in a range of about 252 IU to about 378 IU. In another embodiment of the invention, vitamin D may be present in a range of about 283.5 IU to about 346.5 IU. In a preferred embodiment, vitamin D may be present in the amount of about 315 IU. In a preferred embodiment of the present invention, the form of vitamin D is vitamin D3.

As discussed previously, the antioxidant components of the compositions and methods described herein preferably include vitamin E, selenium, vitamin C, vitamin A, lutein, lipoic acid, and lycopene.

Vitamin E is a fat-soluble vitamin antioxidant found in biological membranes where it protects the phospholipid membrane from oxidative stress. More specifically, alpha-tocopherol, the most abundant and most active form of the vitamin E family, is the principle lipid-soluble, chain breaking antioxidant in tissue and plasma. Recommended Dietary Allowances 99-101 (Nat'l Research Council, 10th ed., 1989) (hereinafter “RDA”). Vitamin E inhibits the oxidation of unsaturated fatty acids by trapping peroxyl free radicals. It is also an antiatherogenic agent, and studies have demonstrated a reduced risk of coronary heart disease with increased intake of vitamin E. Stampfer et al., 328 New Eng. J. Med. 1444-49 (1993). Vitamin E is available in various forms known to those of skill in the art. One embodiment of the compositions utilized by the methods of the present invention may comprise vitamin E in the amount ranging from about 62.5 IU to about 187.5 IU. Another embodiment of the invention may comprise vitamin E in a range of about 100 IU to about 150 IU. In another embodiment of the invention, vitamin E may be present in a range of about 112.5 IU to about 137.5 IU. In a preferred embodiment, vitamin E may be present in the amount of about 125 IU. In a preferred embodiment of the present invention, the form of vitamin E may be d-alpha tocopheryl succinate.

Along with vitamin E, the mineral selenium is a component of the antioxidant enzyme, glutathione peroxidase, which plays a critical role in the control of oxygen metabolism, particularly catalyzing the breakdown of hydrogen peroxide. Burk, 3 Ann. Rev. Nutrition 53-70 (1983). Glutathione peroxidase prevents the generation of free radicals and decreases the risk of oxidative damage to numerous tissues, including the vascular system. Holben, 99 J. Am. Dietary Assoc. 836-43 (1999). Another selenoprotein is the enzyme iodothyronine 5′-diodinase that converts thyroxine (T4) to triiodothyronine (T3). Selenium is available in many forms known to those of ordinary skill in the art.

One embodiment of the compositions utilized by the methods of the present invention may comprise selenium in an amount ranging from about 62.5 μg to about 187.5 μg. In another embodiment, selenium may be present in a range of about 100 μg to about 150 μg. In a further embodiment, selenium may be present in a range of about 112.5 μg to about 137.5 μg. In a preferred embodiment, selenium may be present in an amount of about 125 μg. In another preferred embodiment of the invention, selenium may be in the form of sodium selenate or selenomethionine.

Vitamin C (also known as ascorbic acid) is another antioxidant present in the invention described herein. The major biochemical role of the water-soluble vitamin C is as a co-substrate in metal catalyzed hydroxylations, and it has antioxidant properties in interacting directly with superoxide hydroxyl radicals and singlet oxygen. Vitamin C also provides antioxidant protection for folate and vitamin E, keeping vitamin E in its most potent form. It also enhances the absorption of iron. RDA, at 115. In addition, vitamin C is required for collagen synthesis, epinephrine synthesis, and bile acid formation. Moreover, vitamin C has been implicated in inhibiting atherosclerosis by being present in extracellular fluid of the arterial wall and potentiating nitric oxide activity, thus normalizing vascular function.

One embodiment of the compositions utilized by the methods of the present invention may comprise vitamin C in an amount ranging from about 187.5 mg to about 562.5 mg. In another embodiment, vitamin C may be present in a range of about 300 mg to about 450 mg. In a further embodiment, vitamin C may be present in a range of about 337.5 mg to about 412.5 mg. In a preferred embodiment, vitamin C may be present in an amount of about 375 mg.

Along with vitamins E and C, and selenium, carotenoids are embodied in the present invention. Carotenoids occur naturally in fruits and vegetables. The compositions and methods herein include a cartonoid complex that closely mirrors that found naturally in fruits and vegetables. In particular, the carotenoids of the present invention may preferably include lutein, vitamin A, alpha lipoic acid and lycopene. In particular, lutein is one of the major carotenoids that make up the macular pigment of the eye's retina, and its antioxidant properties protect the eye from light-induced damage and macular degeneration. Berendschot et al., 41 Invest. Ophthalmol. Vis. Sci. 3322-26 (2000). One embodiment of the compositions utilized by the methods of the present invention may comprise vitamin A in an amount ranging from about 375 IU to about 1125 IU. In another embodiment, vitamin A may be present in a range of about 600 IU to about 900 IU. In a further embodiment, vitamin A may be present in a range of about 675 IU to about 825 IU. In a preferred embodiment, vitamin A may be present in an amount of about 750 IU. In another preferred embodiment of the invention, vitamin A may be in the form of beta-carotene or alpha-carotene.

Lutein is also preferably included in the compositions utilized by the methods described herein and is preferably included in an amount distinguished from that included in the mixed carotenoids. Regarding the antioxidant activity of lutein, scientists have demonstrated that lutein is an effective antioxidant capable of scavenging peroxyl radicals and quenching reactive oxygen species. Rapp et al., 41 Invest. Ophthalmol. Vis. Sci. 1200-09 (2000). One embodiment of the compositions utilized by the methods of the present invention may comprise lutein in an amount ranging from about 3.5 mg to about 10.5 mg. In another embodiment, lutein may be present in an range of about 5.6 mg to about 8.4 mg. In a further embodiment, lutein may be present in a range of about 6.3 mg to about 7.7 mg. In a preferred embodiment, lutein may be present in an amount of about 7 mg. Lutein may be in 5% form (lutein 5%).

Lipoic acid is an antioxidant and is preferably included in the compositions and methods of the present invention. Known as the “universal antioxidant,” alpha lipoic acid is both a lipid- and water-soluble antioxidant that works synergistically with other antioxidants in the cell's mitochondria. In addition to working with other antioxidant nutrients, lipoic acid has powerful, pro-antioxidant enzyme properties. Alpha lipoic acid is also a cofactor for several regulatory enzymes, including pyruvate dehydrogenase, and appears to have an effect on glucose transport and utilization. Rudich et al., 42 Diabetologia 949-57 (1999). Alpha lipoic acid also increases tocopherol activity and acts as a metal chelator. Furthermore, alpha lipoic acid improves microvascular perfusion. Haak et al., 108 Experimental & Clinical Endocrinology & Diabetes 168-74 (2000). One embodiment of the compositions utilized by the methods of the present invention may comprise alpha lipotic acid in an amount ranging from about 5 mg to about 15 mg. In another embodiment, alpha lipotic acid may be present in a range of about 8 mg to about 12 mg. In a further embodiment, alpha lipotic acid may be present in a range of about 9 mg to about 11 mg. In a preferred embodiment, alpha lipotic acid may be present in an amount of about 10 mg.

Lycopene is an antioxidant and is similar to other carotenoids. Lycopene protects cells against oxidative damage and is a potent scavenger of oxygen radicals. Lycopene has been linked in various studies to reducing the risk of digestive tract cancer, colon cancer, breast cancer, prostate cancer, and skin cancer. Lycopene has also been shown to lower cholesterol levels and thus reduce the risk of cardiovascular disease. Available at http://www.wholehealthmd.com/refshelf/substances_view/1,1525,803,00.html (last visited 10 Aug. 2005). One embodiment of the compositions utilized by the methods of the present invention may comprise lycopene in an amount ranging from about 1.25 mg to about 3.75 mg. In another embodiment, lycopene may be present in a range of about 2 mg to about 3 mg. In a further embodiment, lycopene may be present in a range of about 2.25 mg to about 2.75 mg. In a preferred embodiment, lycopene may be present in an amount of about 2.5 mg. Lycopene may be in 5% form (lycopene 5%).

Minerals are inorganic, or non-carbon-containing, elements that are critical for healthy physiological processes, and are contemplated in the compositions utilized by the methods of the present invention. For example, minerals act as cofactors for hundreds of enzymes that range from those associated, for example, with food digestion, nucleic acid production, protein synthesis to antioxidant enzymes. One particular mineral, chromium, is essential in healthy insulin function, as it plays a direct role in insulin's interactions at the cellular level. Minerals are inorganic elements that play a crucial role in physiological processes in the body relating to good health. The compositions and methods of the present invention may comprise minerals, and, in a preferred embodiment, comprise one or more of selenium, discussed above, and magnesium, zinc, chromium, copper, and iron.

Magnesium is found primarily in both bone and muscle. Magnesium is an essential component for over 300 enzymes, including enzymes of biosynthetic pathways, glycolysis, protein synthesis, transketolase reactions, and membrane transport. Magnesium is also involved in the formation of cAMP, a cytosolic second messenger that plays a role in cell signaling mechanisms. In addition, magnesium functions both synergistically and antagonistically with calcium in neuromuscular transmission. RDA, at 188. Specifically, magnesium is critical for the maintenance of electrochemical potentials of nerve and muscle membranes and the neuromuscular junction transmissions, particularly important in the heart. Not surprisingly, magnesium deficiency is tied to cardiovascular disease and hypertension. Agus et al., 17 Crit. Care Clinics 175-87 (2001). Indeed, oral magnesium therapy improves endothelial function in patients with coronary disease. Shechter et al., 102 Circulation 2353-58 (2000). Yet, most individuals in the U.S. receive only about seventy-five percent of the magnesium they need from their diets. Magnesium is available in a variety of salts. One embodiment of the compositions utilized by the methods of the present invention may comprise magnesium in an amount ranging from about 17.5 mg to about 52.5 mg. In another embodiment, magnesium may be present in a range of about 28 mg to about 42 mg. In a further embodiment, magnesium may be present in a range of about 31.5 mg to about 37.5 mg. In a preferred embodiment, magnesium may be present in an amount of about 35 mg. In a preferred embodiment, magnesium may be present in the form of magnesium oxide.

Zinc plays a role in numerous metabolic activities such as nucleic acid production, protein synthesis, and development of the immune system. There are more than 200 zinc metalloenzymes including aldolase, alcohol dehydrogenase, RNA polymerase, and protein kinase C. Zima et al., 17 Blood Purif. 182-86 (1999). Moreover, zinc stabilizes RNA and DNA structures, forms zinc fingers in nuclear receptors, and is a component of chromatin proteins involved in transcription and replication. Zinc is available in many forms, such as zinc oxide and zinc sulfate. One embodiment of the compositions utilized by the methods of the present invention may comprise zinc in an amount ranging from about 17.5 mg to about 52.5 mg. In another embodiment, zinc may be present in a range of about 28 mg to about 42 mg. In a further embodiment, zinc may be present in a range of about 32.5 mg to about 37.5 mg. In a preferred embodiment, zinc may be present in an amount of about 35 mg. In a preferred embodiment, zinc may be present in the form of zinc oxide.

The trace mineral chromium harmonizes with insulin at the cellular level to optimize the release of energy from glucose, as well as maintaining proper cellular lipid or fat metabolism. Specifically, chromium increases insulin binding to cells, insulin receptor number, and activates the insulin receptor kinase leading to increased insulin sensitivity. Several studies suggest that adequate chromium levels are needed for optimal glycemic control. See, e.g., Anderson et al., 26 Diabetes Metabolabolism 22-27 (2000); Vincent, 130 J. Nutrition 715-18 (2000). The concentration of chromium declines with age, and coronary artery disease appears to be associated with low levels of chromium. RDA, at 241. Yet, ninety percent of adults in the U.S. consume less than the recommended minimum amount of chromium. Chromium is available in various forms known to those skilled in the art, such as chromium chloride, chromium sulfate, chromium potassium sulfate, and chromium picolinate. One embodiment of the compositions utilized by the methods of the present invention may comprise chromium in an amount ranging from about 75 μg to about 225 μg. In another embodiment, chromium may be present in a range of about 120 μg to about 170 μg. In a further embodiment, chromium may be present in a range of about 135 μg to about 165 μg. In a preferred embodiment, chromium may be present in an amount of about 150 μg. In a preferred embodiment, chromium may be present in the form of chromium chloride or picolinate.

Copper is a component of several enzymes associated with numerous physiological functions, including, for example, oxidase enzymes, such as cytochrome c oxidase, and cytosolic superoxide dismutase. RDA, at 224. In particular, copper is a cofactor of lysyl oxidase, which is critical for lysine cross-linking in collagen and elastin. Copper acts as an antioxidant, and promotes the synthesis of melanin and catecholamines. In addition, copper is present in the blood as ceruloplasmin which is involved in oxidizing iron prior to transport to the plasma. Copper is available in multiple forms, such as cupric oxide, copper sulfate, cupric acetate, and alkaline copper carbonate. One embodiment of the compositions utilized by the methods of the present invention may comprise copper in an amount ranging from about 0.5 mg to about 1.5 mg. In another embodiment, copper may be present in a range of about 0.8 mg to about 1.2 mg. In a further embodiment, copper may be present in a range of about 0.9 mg to about 1.1 mg. In a preferred embodiment, copper may be present in an amount of about 1.0 mg. In a preferred embodiment, copper may be present in the form of cupric sulfate or gluconate.

Iron is an essential mineral in the human body. The main function of iron is to help carry oxygen from the lungs to the muscles to other organs in the body. When iron levels are low in the body, oxygen consumption slows down. When depleted, oxygen circulates more slowly and fatigue, irritability and headaches may occur. If iron deficiency becomes significant, this can lead to anemia. Several select groups are more at risk for developing iron deficiencies. Women are more likely to develop low iron in their blood because of the loss of red blood cells during menstruation. Pregnant women must also be careful to consume enough iron. Further, adolescents may be lacking in iron due to rapid growth processes. Finally, athletes may be at risk because exercising regularly can cause iron loss through perspiration. Supplementation with iron can help maintain iron levels and can help prevent the effects of low iron in the blood, including anemia. Available at http://sparkpeople.com/resource/nutrition_articles.asp?id=48 (last visited 10 Aug. 2005); http://adam.about.com/reports/000057_{—}6.htm (last visited 10 Aug. 2005).

One embodiment of the compositions utilized by the methods of the present invention may comprise iron in an amount ranging from about 6.5 mg to about 19.5 mg. In another embodiment, iron may be present in a range of about 10.4 mg to about 15.6 mg. In a further embodiment, iron may be present in a range of about 11.7 mg to about 14.3 mg. In a preferred embodiment, iron may be present in an amount of about 13 mg. In a preferred embodiment, iron may be present in the form of iron ferronyl.

The active ingredients are available from numerous commercial sources, and in several active forms or salts thereof, known to those of ordinary skill in the art. Hence, the compositions and methods of the present invention are not limited to comprising or using any particular form of the vitamin or mineral ingredient described herein.

Nutrition is a constantly evolving health science. Nearly as proliferative as research findings correlating nutrients and disease prevention are findings demonstrating that supplementation with some nutrients can be counter-productive to the health needs of specific populations. In a specific embodiment, the compositions and methods of the present invention may be substantially free of other added vitamins and minerals.

The compositions of the present invention are preferably administered in amounts to patients that provide the supplementation required to alleviate the vitamin deficiencies associated with anemia, cardiovascular disease and cancer as well as provide vitamin and mineral supplementation to alleviate physiologically stressful conditions on the body. A preferred dosage of the compositions of the present invention may consist of one or more caplets for human oral consumption. If more than one caplet is used, each individual caplet may be identical to the other caplets, or each may contain only some of the ingredients of the composition, so that the combination of the different caplets comprises a composition of the present invention.

A specific embodiment of the present invention may comprise swallowable compositions. Swallowable compositions are well known in the art and are those that do not readily dissolve when placed in the mouth and may be swallowed whole without any chewing or discomfort. In a specific embodiment of the present invention, the swallowable compositions may have a shape containing no sharp edges and a smooth, uniform and substantially bubble free outer coating.

To prepare the swallowable compositions of the present invention, each of the active ingredients may be combined in intimate admixture with a suitable carrier according to conventional compounding techniques. In a specific embodiment of swallowable compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the swallowable compositions of the present invention include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia. Pharmaceutical carriers and formulations for swallowable compounds are well known to those of ordinary skill in the art. See generally, e.g., Wade & Waller, Handbook of Pharmaceutical Excipients (2nd ed. 1994).

Disintegrants also may be included in the compositions of the present invention in order to facilitate dissolution. Disintegrants, including permeabilising and wicking agents, are capable of drawing water or saliva up into the compositions which promotes dissolution from the inside as well as the outside of the compositions. Such disintegrants, permeabilising and/or wicking agents that may be used in the present invention include by way of example and without limitation, starches such as corn starch, potato starch, pre-gelatinized and modified starches thereof, cellulosic agents such as Ac-di-sol, montmorrilonite clays, cross-linked PVP, sweeteners, bentonite, microcrystalline cellulose, croscarmellose sodium, alginates, sodium starch glycolate, gums such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents such as hydroxymethylcellulose, hydroxypropylcellulose and hydroxyopropylmethylcellulose.

Finally, dissolution of the compositions may be facilitated by including relatively small particles sizes of the ingredients used.

In addition to those described above, any appropriate fillers and excipients may be utilized in preparing the swallowable compositions of the present invention so long as they are consistent with the objectives described herein. For example, binders are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium, and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol, and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, and polyols of less than 13 carbon atoms, such as mannitol, xylitol, sorbitol, maltitol, and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly(ethylene glycol), glyceryl behenate, stearyl fumarate, and others known to those of ordinary skill in the art.

Glidants improve the flow of powder blends during manufacturing and minimize composition weight variation. Glidants that may be used in the present invention include by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions of the present invention. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art. Coloring agents also can include pigments, dyes, tints, titanium dioxide, natural coloring agents such as grape skin extract, beet red powder, beta carotene, annato, carmine, turmeric, paprika, and others known to those of ordinary skill in the art. It is recognized that no colorant is required in the nutritional supplement compositions described herein.

If desired, compositions may be sugar coated or enteric coated by standard techniques. The unit dose forms may be individually wrapped, packaged as multiple units on paper strips or in vials of any size, without limitation. The swallowable, chewable or dissolvable compositions of the invention may be packaged in unit dose, rolls, bulk bottles, blister packs and combinations thereof, without limitation.

The swallowable compositions of the present invention may be prepared using conventional methods and materials known in the pharmaceutical art. For example, U.S. Pat. Nos. 5,215,754 and 4,374,082 relate to methods for preparing swallowable compositions. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in Wade & Waller, supra. All active ingredients, fillers and excipients are commercially available from companies such as Aldrich Chemical Co., FMC Corp, Bayer, BASF, Alexi Fres, Witco, Mallinckrodt, Rhodia, ISP, and others.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

Without further elaboration, it is believed that one skilled in the art, using the preceding description, can utilize the present invention to the fullest extent. The following example is illustrative only, and not limiting of the reminder of the disclosure in any way whatsoever.

### Example 1

A composition of the following formulation was prepared in caplet form, including the appropriate excipients, by standard methods known to those of ordinary skill in the art:

| | | |
|---|---|---|
| Vitamin A | 750 | IU |
| Vitamin E | 125 | IU |
| Vitamin D₃ | 315 | IU |
| Vitamin C (Ascorbic Acid) | 375 | mg |
| Vitamin B₁ (Thiamine HCl) | 25 | mg |
| Vitamin B₂ (Riboflavin) | 3.4 | mg |
| Niacin (Niacinamide) | 35 | mg |
| Folic Acid | 1.25 | mg |
| Vitamin B₆ (Pyridoxine HCl) | 35 | mg |
| Biotin | 75 | μg |
| Pantothenic Acid (Calcium Pantothenate) | 5 | mg |
| Vitamin B₁₂ (Cyanocobalamin) | 70 | μg |
| Magnesium (Magnesium Oxide) | 35 | mg |
| Zinc (Zinc Oxide) | 35 | mg |
| Selenium (Sodium Selenate) | 1250 | μg |
| Chromium (Chromium Chloride) | 150 | μg |
| Copper (Cupric Sulfate) | 1 | mg |
| Iron (Iron Ferronyl, Micronized) | 13 | mg |
| Alpha Lipoic Acid | 10 | mg |
| Lutein 5% | 7 | mg |
| Lycopene 5% | 2.5 | mg |

### Example 2

A study is undertaken to evaluate the effectiveness of the composition of the present invention in the treatment of patients. The objective of the study is to determine whether oral intake of the composition results in an improvement of the nutritional status of the patient, either therapeutically or prophylacticly.

A double-blind, placebo controlled study is conducted over a twelve-month period. A total of sixty subjects (30 men and 30 women), aged 40 to 85 years, suffering from dietary restrictions or a disease state such as cancer, cardiovascular disease or anemia or a propensity or disposition to such a disease state are chosen for the study. An initial assessment of nutritional status is conducted utilizing methods such as the peroxide hemolysis test to assess vitamin E deficiency, measurement of erythrocyte transketolase activity to determine thiamine levels, determination of erythrocyte glutathione reductase activity to assess riboflavin status, and high performance liquid chromatography to directly measure PLP and pyridoxine levels.

The sixty subjects are separated into two separate groups of fifteen men and fifteen women. In the first group, each subject is administered 1 to 2 caplets, daily, of the composition as described in Example 1. In the second group (control), each subject is administered 1 to 2 placebo caplets, daily.

An assessment of nutritional status for each subject is measured at one-month intervals for a twelve month period as described above and the data is evaluated using multiple linear regression analysis and a standard students t-test. In each analysis the baseline value of the outcome variable is included in the model as a covariant. Treatment by covariant interaction effects is tested by the method outlined by Weigel & Narvaez, 12 Controlled Clinical Trials 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 16, 12, and 8 weeks, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in the nutritional status is preferably observed in the treated subjects upon completion of the study over the controls. The study may also look at the progression of the disease state, or the prevention or delay of a disease or disease state, or the reduction of the severity of a disease. The differences between nutritional state or the progression of the disease state, or the prevention or delay of a disease or disease state, or the reduction of the severity of a disease, between the treated subjects and controls are preferably statistically significant and or observable by clinical or other tests or evaluations. Therefore, the study confirms that oral administration of the composition of the present invention is effective as a nutritional supplement, either therapeutically or prophylacticly, for example, in preventing the severity or delaying or preventing the onset of a disease.

While there has been described what is presently believed to be the preferred embodiments of the present invention, other and further modifications and changes may be made without departing from the spirit of the invention. All further and other modifications and changes are included that come within the scope of the invention as set forth in the claims. The disclosure of all publications cited above are expressly incorporated by reference in their entireties to the same extent as if each were incorporated by reference individually.

1. A method comprising administering a composition comprising vitamin A, vitamin E, vitamin D, vitamin C, thiamine, riboflavin, niacin, folic acid, pyridoxine, biotin, pantothenic acid, cobalamin, magnesium, manganese, zinc, selenium, chromium, copper, iron, alpha lipoic acid, lutein, and lycopene, wherein said composition is substantially free of other added vitamins and added minerals.

2. The method of claim 1, further comprising pharmaceutically acceptable carriers.

3. The method of claim 2, wherein said pharmaceutically acceptable carriers are selected from one or more of the group consisting of binders, diluents, lubricants, glidants, colorants, emulsifiers, disintegrants, starches, water, oils, alcohols, preservatives and sugars.

4. The method of claim 1, wherein said vitamin A is selected from the group consisting of alpha-carotene and beta-carotene.

5. The method of claim 1, wherein said vitamin A is present in the range of about 675 IU to about 825 IU.

6. The method of claim 1, wherein said vitamin E comprises d-alpha tocopheryl succinate.

7. The method of claim 1, wherein said vitamin E is present in the range of about 112.5 IU to about 137.5.

8. The method of claim 1, wherein said vitamin D comprises D₃.

9. The method of claim 1, wherein said vitamin D is present in the range of about 283.5 IU to about 346.5 IU

10. The method of claim 1, wherein said vitamin C is present in the range of about 337.5 mg to about 412.5 mg.

11. The method of claim 1, wherein said thiamine is selected from the group consisting of thiamine HCl and thiamine mononitrate.

12. The method of claim 1, wherein said thiamine is present in the range of about 22.5 mg to about 27.5 mg.

13. The method of claim 1, wherein said riboflavin is present in the range of about 3.06 mg to about 3.74 mg.

14. The method of claim 1, wherein said niacin comprises niacinamide.

15. The method of claim 1, wherein said niacin is present in the range of about 31.5 mg to about 37.5 mg.

16. The method of claim 1, wherein said folic acid is selected from the group consisting of B₉, folacin, metafolin, folate, (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof.

17. The method of claim 1, wherein folic acid is present in the range of about 1.125 mg to about 1.375 mg.

18. The method of claim 1, wherein said pyridoxine comprises pyridoxine HCl.

19. The method of claim 1, wherein said pyridoxine is present in the range of 31.5 mg to about 37.5 mg.

20. The method of claim 1, wherein said biotin is present in the range of about 67.5 μg to about 82.5 μg.

21. The method of claim 1, wherein said pantothenic acid comprises calcium pantothenate.

22. The method of claim 1, wherein said pantothenic acid is present in the range of about 4.5 mg to about 5.5 mg.

23. The method of claim 1, wherein said cobalamin comprises cyanocobalamin.

24. The method of claim 1, wherein said cobalamin is present in the range of about 63 μg to about 77 μg.

25. The method of claim 1, wherein said magnesium comprises magnesium oxide.

26. The method of claim 1, wherein said magnesium is present in the range of about 31.5 mg to about 37.5 mg.

27. The method of claim 1, wherein said zinc comprises zinc oxide.

28. The method of claim 1, wherein said zinc is present in the range of about 32.5 mg to about 37.5 mg.

29. The method of claim 1, wherein said selenium is selected from the group consisting of sodium selenate and selenomethionine.

30. The method of claim 1, wherein said selenium is present in the range of about 112.5 μg to about 137.5 μg.

31. The method of claim 1, wherein said chromium is selected from the group consisting of chromium chloride and picolinate.

32. The method of claim 1, wherein said chromium is present in the range of about 135 μg to about 165 μg.

33. The method of claim 1, wherein said copper is selected from the group consisting of cupric sulfate and gluconate.

34. The method of claim 1, wherein said copper is present in the range of about 0.9 mg to about 1.1 mg.

35. The method of claim 1, wherein said iron comprises iron ferronyl.

36. The method of claim 1, wherein said iron is present in the range of about 11.7 mg to about 14.3 mg.

37. The method of claim 1, wherein said alpha lipoic acid is present in the range of about 9 mg to about 11 mg.

38. The method of claim 1, wherein said lutein is present in the range of about 6.3 mg to about 7.7 mg.

39. The method of claim 1, wherein said lycopene is present in the range of about 2.25 mg to about 2.75 mg.

40. The method of claim 1 wherein said composition comprises about 675 IU to about 825 IU vitamin A, about 112.5 IU to about 137.5 IU vitamin E, about 283.5 IU to about 346.5 IU vitamin D, about 337.5 mg to about 412.5 mg vitamin C, about 22.5 mg to about 27.5 mg thiamine, about 3.06 mg to about 3.74 mg riboflavin, about 31.5 mg to about 37.5 mg niacin, about 1.125 mg to about 1.375 mg folic acid, about 31.5 mg to about 37.5 mg pyridoxine, about 67.5 μg to about 82.5 pg biotin, about 4.5 mg to about 5.5 mg pantothenic acid, about 63 μg to about 77 μg cobalamin, about 31.5 mg to about 37.5 mg magnesium, about 32.5 mg to about 37.5 mg zinc, about 112.5 μg to about 137.5 μg selenium, about 135 μg to about 165 μg chromium, about 0.9 mg to about 1.1 mg copper, about 11.7 to about 14.3 mg iron, about 9 mg to about 11 mg alpha lipoic acid, about 6.3 mg to about 7.7 mg lutein, and about 2.25 mg to about 2.75 mg lycopene.

41. The method of claim 1, wherein said composition comprises about 750 IU vitamin A, about 125 IU vitamin E, about 315 IU vitamin D, about 375 mg vitamin C, about 22 mg thiamine, about 3.4 mg riboflavin, about 35 mg niacin, about 1.25 mg folic acid, about 35 mg pyridoxine, about 75 μg biotin, about 5 mg pantothenic acid, about 70 μg cobalamin, about 35 mg magnesium, about 35 mg zinc, about 125 μg selenium, about 150 μg chromium, about 1.0 mg copper, about 13 mg iron, about 10 mg alpha lipoic acid, about 7 mg lutein, and about 2.5 mg lycopene.

42. The method of claim 1, wherein said composition is administered to a patient.

43. The method of claim 42, wherein said composition is administered to said patient orally.

44. The method of claim 42, wherein said composition is administered to said patient to prevent, treat and/or alleviate the occurrence or negative effects of vitamin and mineral deficiencies.

45. The method of claim 42, wherein said composition is administered to said patient to treat and/or alleviate the occurrence or negative effects of one or more of the group consisting of cancer, cardiovascular disease, and anemia.

46. The method of claim 44, wherein said patient is selected from one or more of the group consisting of premenopausal women, dieters, high-impact endurance athletes, infants, children, adolescents, and pregnant women.

47. A method comprising administering to a patient a composition comprising vitamin A, vitamin E, vitamin D, vitamin C, thiamine, riboflavin, niacin, folic acid, pyridoxine, biotin, pantothenic acid, cobalamin, magnesium, manganese, zinc, selenium, chromium, copper, iron, alpha lipoic acid, lutein, and lycopene, wherein said composition is substantially free of other added vitamins.
